(19) United States
(12) Patent Application Publication
(10) Pub. No.: US 2006/0211926 A1
(43) Pub. Date: Sep. 21, 2006

(54) Non-invasive Raman measurement apparatus with broadband spectral correction

(76) Inventors: Dejin Yu, Fremont, CA (US); Wei Yang, Fremont, CA (US)

Correspondence Address: STALLMAN & POLLOCK LLP, 353 SACRAMENTO STREET, SUITE 2200, SAN FRANCISCO, CA, 94111, US

(21) Appl. No.: 11/085,751

(22) Filed: Mar. 21, 2005

(51) Int. Cl. A61B 5/00

(52) U.S. Cl. 600/316; 600/322

(57) ABSTRACT

The present invention discloses a method and apparatus in relation to non-invasive measurement of human blood analytes such as glucose using Raman Spectroscopy. The method is intended to correct for errors associated with varying skin conditions. An approach is described for generating a skin/tissue function that accounts for light energy dissipation due to skin and tissue interferences such as color, thickness, sites, oily, turbid, and surface roughness, etc. The measured skin/tissue function is utilized to correct Raman spectra, thus removing uncertainty and ambiguity coming from skin conditions. The method comprises measuring incident broadband spectrum and diffuse reflectance spectrum, and from them calculating the skin-tissue function.

### TECHNICAL FIELD OF THE INVENTION

This invention in general relates to method and technique for correcting optical signals of spectroscopic technologies used for non-invasive measurement of concentrations of human blood constitutes, and in particular, for monitoring the blood glucose levels in vivo for diabetes.

### BACKGROUND OF THE INVENTION

Currently, daily blood glucose self-monitoring for diabetes patients can only be done through the use of invasive method that requires drawing blood from patients. It is painful and inconvenient since the skin has to be lanced into the soft tissue in order to collect the blood sample for the periodic measurements, typically 6-8 times a day. It is the same routine for the diabetics in order to provide feedback for insulin dosing and other management. Clinical studies have shown that a tight glucose control with frequent glucose measurements leads to a substantial decrease in the long-term complications of diabetes.

There has been growing interest and high desire to develop a non-invasive method and apparatus that is suitable for frequently or continuously monitoring glucose levels without drawing blood. In the last decade, various attempts have been made to measure blood glucose level non-invasively (or in vivo), mainly using spectroscopic technologies in which the concentration of analytes is determined from spectral information through light-blood interaction. These techniques include visible and near-infrared (NIR) spectroscopy, mid-infrared (MIR) spectroscopy, infrared (IR) spectroscopy, diffuse reflectance spectroscopy, fluorescence spectroscopy, polarimetry, scattering changes, photo-acoustic spectroscopy, and Raman scattering through human eyes, etc. Most recently Raman lightwave technology through human skin (U.S. Pat. No. 6,167,290) has been employed to measure glucose non-invasively. These optical methods open up an opportunity for developing a new generation of glucose meters to detect blood glucose without drawing a blood sample.

To date, all of these optical methods suffer a number of technical difficulties though Raman scattering is believed to be the most promising technology in this direction. It is worth noting that all non-invasive lightwave technologies, such as infrared absorption spectroscopy and its variants, both NIR and MIR, diffuse reflectance spectroscopy, fluorescence spectroscopy, and Raman spectroscopy, involve the penetration of excitation light energy into skin and, after the light-blood interaction, the derived signal comes through the skin back into free space. A technical issue that prevents the non-invasive glucose concentration from being accurately measured is the energy dissipation process arising from the skin and tissues as light propagates. The direct consequence is poor repeatability and reproducibility of optical measurements. For example, different skin color and thickness will lead to different light power loss, and different types of skin conditions (e.g., roughness and moisture) will create different degrees of interference, such as different scattering and absorption strength. This practical problem has so far not been addressed.

The intention of the present invention is to provide an optical method and technique that accounts for interference from the skin and tissues. The optical signals of a particular spectroscopic technology can be corrected, which is used for precise non-invasive measurement of concentrations of human blood constituents such as glucose.

### SUMMARY OF THE INVENTION

The invention generally provides an optical method and technique to correct optical signal deformation due to optical absorption and scattering arising from skin and tissue. The associated apparatus is designed for non-invasively measuring concentrations of analytes, preferably glucose, from human blood through the skin using a lightwave technique such as Raman Spectroscopy.

It is our object of the present invention to provide an optical method and apparatus to measure in real-time skin/tissue function that is used to account for signal energy loss as it propagates within tissues when used in Raman lightwave system for non-invasive monitoring of blood glucose levels from human subjects without drawing blood.

Another object of the present invention is to provide a method to correct signal distortion in real-time due to light absorption as it propagates within tissues, including the skin.

Still another object of the present invention is to provide a method to correct signal distortion due to light scattering as it propagates within tissues and on the skin surface.

Yet another object of the present invention is to provide an algorithm to correct optical signals used for non-invasively measuring concentrations of analytes, preferably glucose, from human blood through the skin using a lightwave technique such as Raman Spectroscopy.

The present invention discloses a skin/tissue function and its important role for non-invasive spectroscopic technologies used for measuring the concentrations and physiological levels of analytes from human blood through the skin. The skin/tissue function disclosed in the present invention can be employed to account for signal energy dissipation process and remove the uncertainty of signal analysis in all spectroscopic technologies described above, including, for example, infrared absorption spectroscopy and its variants, diffuse reflectance spectroscopy, fluorescence spectroscopy, Fourier-transform infrared (FTIR) spectroscopy, and Raman spectroscopy.

The disclosed skin/tissue function is defined and measured over the particular spectral range of interest. For example, when used for Raman lightwave system, the spectral range is consistent with the Raman wavelengths, i.e., from the pump wavelength to the maximum Raman shift.

In one embodiment, the skin/tissue function is measured at one specific wavelength. The corresponding optical system and apparatus is relatively simple. The preferred wavelength is selected at the center of the spectral range.

In another embodiment, the skin/tissue function is measured at a few discrete wavelengths. Typically, three wavelengths are used, one at lower end, the second one at the middle and the remaining one at the upper end. For a narrow spectral range, the skin/tissue function obtained over these three wavelengths gives a good estimation.

In still another embodiment, the skin/tissue function is preferably measured over a continuous spectral region. To this end, a broadband light source is used to obtain the correction signals.

The aforementioned method and apparatus for skin/tissue function can be applied to various spectroscopic systems for non-invasively monitoring levels of blood analytes through the skin. Further objects and advantages of the subject invention will be apparent from the following drawings and detailed description of the preferred embodiments.

### BRIEF DESCRIPTION OF THE DRAWINGS

These, as well as other features of the present invention, will become more apparent upon reference to the drawings wherein:

FIG. 1 is a block diagram schematically illustrating a basic configuration of the Raman spectroscopic apparatus used for non-invasively measuring blood glucose level in accordance with the prior art.

FIG. 2 is a preferred embodiment of the present invention for measuring the skin/tissue function.

FIG. 3 illustrates a simplified model to show the function of the defined skin/tissue function.

FIG. 4 shows another preferred embodiment of the present invention for measuring the skin/tissue function.

FIG. 5 illustrates two examples of skin/tissue function, measured on human index and middle fingers.

### DETAILED DESCRIPTION OF THE PREFERRED EMBODIMENTS

The present invention provides a method and apparatus for accounting for the influence of skin/tissue along with signal processing used to non-invasively measure the concentrations and physiological levels of analytes from human blood through the skin. The invention is described based on Raman spectroscopy, but it can be applied to other lightwave methods including near-infrared spectroscopy, mid-infrared spectroscopy, infrared spectroscopy, reflectance spectroscopy, fluorescence spectroscopy, polarization changes, scatter changes, and photo-acoustic spectroscopy, but is not limited to.

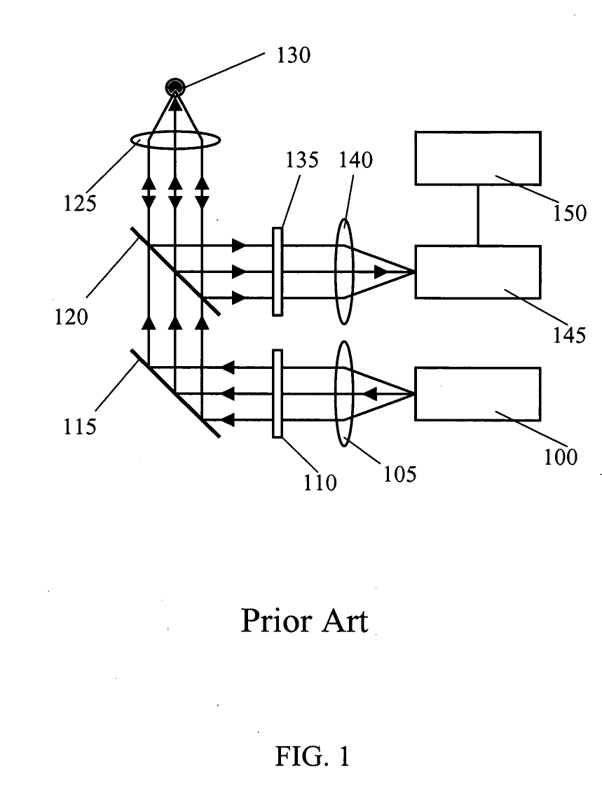

Referring now to the drawings, FIG. 1 illustrates a simplified Raman configuration of the apparatus used for non-invasive measurement of blood glucose level in accordance with the prior art (U.S. Pat. No. 6,167,290). It consists of five parts: 1) excitation laser 100, 2) Raman spectrometer 145, 3) light excitation and collection unit, and 4) data processing unit 150. The CW excitation laser beam is generated from a laser 100, preferably semiconductor laser operated at 750-1000 nm, collimated by a lens 105, filtered by a bandpass filter 110, reflected by a mirror 115, and finally focused by a lens 125 onto the finger 130. The optical elements 100, 105, 110, 115, and 125 form the light excitation unit. The backscattered Raman light from the analytes within 130 through the skin is collected and collimated by the lens 125, reflected by the beam splitter 120, filtered by a notch filter 135 and then focused by a lens 140 onto the entrance slit of Raman spectrometer 145. The optical elements 125, 120, 135, and 140 form the light collection unit. The dispersed Raman spectra are recorded by the detector array, preferably a charge-coupled device (CCD) and transferred to the data processing unit 150 for processing and analysis. In FIG. 1, the bandpass filter 110 generates a narrow band light at a single wavelength λ₀. The notch filter 135 allows Raman-shifted components to pass, but blocks the Rayleigh scattering at pump wavelength λ₀.

Further information about the use of Raman Spectroscopy for measuring constituents in the body can be found in the following patents and patent applications, each of which is incorporated herein by reference: U.S. Pat. No. 6,167,290 (Yang), U.S. Pat. No. 5,481,113 (Dou); U.S. Pat. No. 5,553,616 (Ham); U.S. Pat. No. 5,615,763 (Burger), U.S. Pat. No. 6,151,522 (Alfano), U.S. Pat. No. 5,243,983 (Tarr), and 2003/0208169 (Chaiken).

There exists a practical issue when the Raman apparatus shown in FIG. 1 is directly used to measure Raman scattering spectra through the skin, from which the concentrations and physiological levels of analytes from human blood are analyzed. As described above, many physical processes tend to deform the Raman measurements. These processes and phenomena include, but are not limited to, skin/tissue/blood absorption, Mie scattering, surface scattering, skin colors, skin thickness, skin cleanness, skin sites, and other effects. These processes seriously decrease repeatability and reproducibility of Raman signal measurements, thus generating enormous error in glucose prediction.

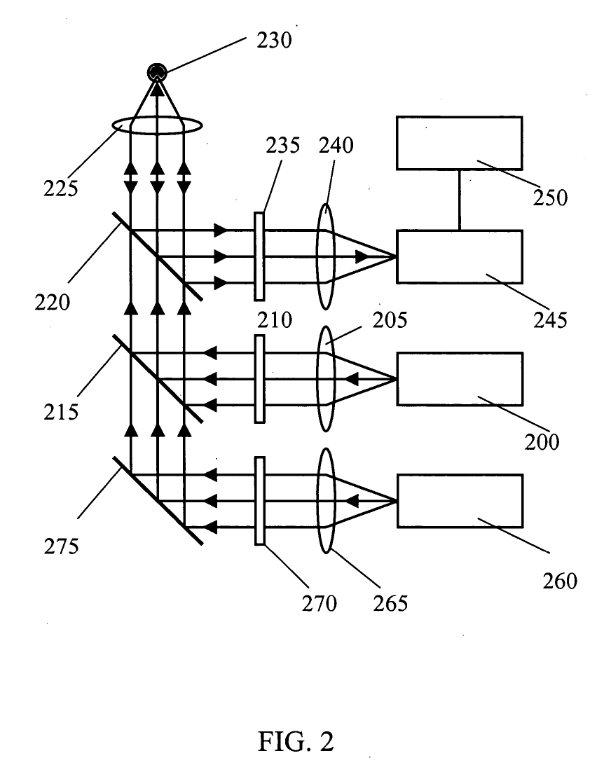

To precisely acquire Raman signal spectra and reduce calculation error for a glucose measurement, an optical apparatus and method is desirable to dynamically measure the effects from skin and tissue and in real-time correction Raman signals. FIG. 2 illustrates the present invention as a new Raman system for this purpose. Apart from the basic optical geometry described in FIG. 1, an additional optical system is added. The element 260 is a broadband light source whose wavelength range covers the pump wavelength and interested Raman shifted wavelengths. The element 270 is a bandpass filter whose transmission band is from the pump wavelength to the longest wavelength in selected Raman scattering. The broadband light from 260 is collimated by an optical system 265, filtered by 270, reflected by 275, and focused by 225 onto the laser-tissue interaction region within the tissue. The illumination power is below the Raman threshold since it is a broadband white light source, rather than a laser.

The generated reflectance signals are collected by the lens 225, reflected by 220, and then focused by 240 onto Raman spectrometer 245. Note that when the broadband light is applied, the laser should be blocked, and vice versa. This requires the mirror 115 in FIG. 1 to be replaced by an optical switch 215. In the simplest arrangement, the element 215 is a movable mirror. When it is in the position as shown in FIG. 2, it blocks the broadband light while reflecting the laser beam. Otherwise, if it is removed, the laser beam cannot be reflected, but the broadband light is allowed to pass. The optical switch may be a mechanical switch that allows one beam to pass and block the other. In another embodiment, two shutters can be used to alternatively block laser or broadband light.

Since the intensity of the light emitted from the broadband source is not constant over the selected wavelength range, it is desirable to normalize the reflectance measurement with a measurement of the light incident on the sample. In the FIG. 2 embodiment, this can be accomplished by rotating the mirror 220 by 90° around an axis normal to the optical path plane to direct the light emitted from the broadband source directly to the spectrometer 245.

By using the above optical apparatus disclosed in the present invention, a skin/tissue function is defined and measured. This characteristic function is defined as

$$f ( λ ) = \frac{P ( λ )}{{P}_{0} ( λ )} \qquad ( 1 )$$

where P(λ) is the reflectance spectrum from skin and tissue, and P₀(λ) is the incident spectrum of broadband light.

The function and role of the skin/tissue function ƒ(λ) is to factor out the energy dissipation caused by various processes mentioned above. Suppose that the measured Raman spectrum is R(λ). It varies not only with glucose concentration but also with variations in the skin and tissue conditions for different people. From the measured skin/tissue function ƒ(λ) and measured Raman spectrum R(λ) and noting that both the pump and Raman scattering signals travel in a single path through the tissue medium, while the broadband light travels both into and out of the tissue medium, a new corrected Raman spectrum R₀(λ) can be defined as

$${R}_{0} ( λ ) = \frac{R ( λ )}{\sqrt{f ( λ )}} \qquad ( 2 )$$

The resulting corrected “Raman spectrum” R₀(λ) is equivalent to the spectrum that would be obtained if it was measured in the absence of these energy dissipation processes.

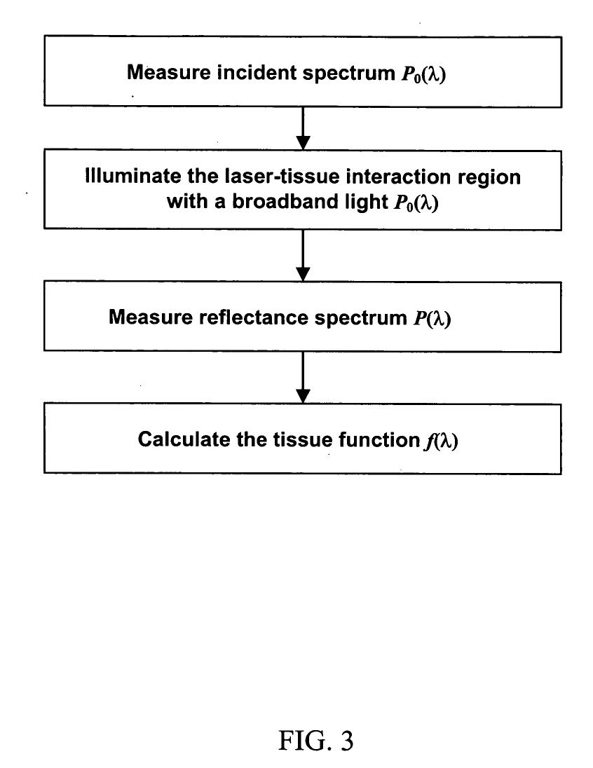

In one preferred embodiment, the skin/tissue function is measured using procedures illustrated in FIG. 3. It involves the following steps:
1. Measure the incident spectrum P₀(λ). It is used as a reference at each wavelength.
2. Illuminate the laser-tissue interaction region with the light P₀(λ). This will generate a reflectance spectrum from skin and tissue.
3. Measure the reflectance spectrum P(λ).
4. Calculate the skin/tissue function f(λ) according to Equation (1).

The skin tissue function f(λ) is then used to correct the measured Raman spectral response, using for example, equation (2). In the preferred embodiment, when broadband light is used to generate the skin/tissue function f(λ), the correction is performed for all wavelength components over the whole selected wavelength range.

While correction on a wavelength by wavelength basis will provide the greatest accuracy, some measure of improvement can be obtained using only a few wavelengths or even a single wavelength. In a simple case, the correction measurements taken at one or a few wavelengths can be applied across the entire spectral range. In a more sophisticated method, a modeling approach could be developed which would define a theoretical skin/tissue function over a large range of wavelengths based on a subset of measurements.

Once the corrected function is obtained, a determination of the concentration of the constituent or analyte is performed in accordance with prior methods. Some of those methods are described in the above cited patents. The current approach preferred by the inventors is described in our copending applications which are incorporated herein by reference: Ser. Nos. 10/914,761, filed on Aug. 9, 2004; Ser. No. 10/940,791 and 10/940,097 both filed on Sep. 14, 2004.

As noted above, the subject correction approach can be used for measurement systems other than Raman Spectroscopy. In particular, where measurements are made with other spectroscopic approaches in which light signals are required to travel within tissues, they can be corrected with the skin/tissue function f(λ).

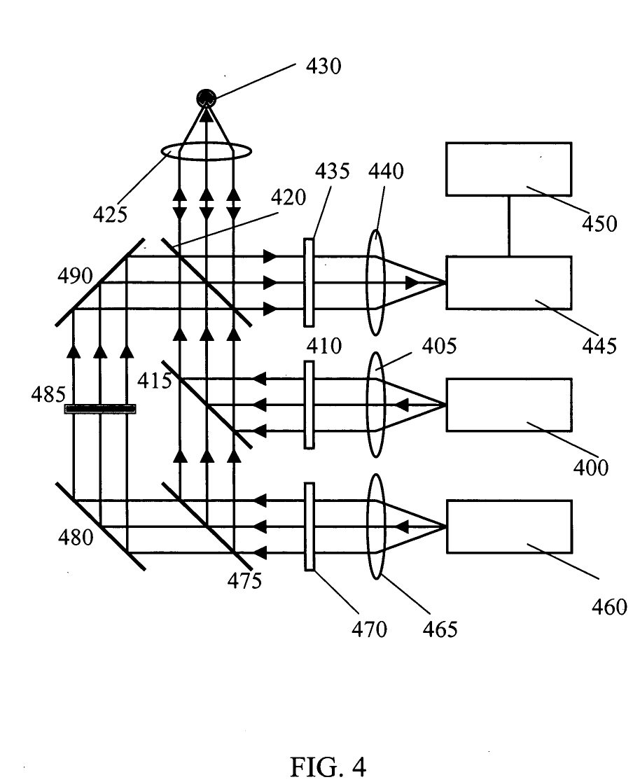

As noted above, measuring the incident spectrum P₀(λ) requires directing the incident broadband light beam to the Raman spectrometer 245. This can be done by moving one or more optical elements as discussed above with respect to FIG. 2. FIG. 4 illustrates another approach that includes an additional optical path that allows the broadband light beam to travel to the Raman spectrometer 245. In FIG. 4, both 480 and 490 are mirrors, and the element 485 is an optical blocker that can be at “on” and “off” states for opening and blocking the optical path. The operation of each optical element should be coordinated and controlled by electronics system according to their functions.

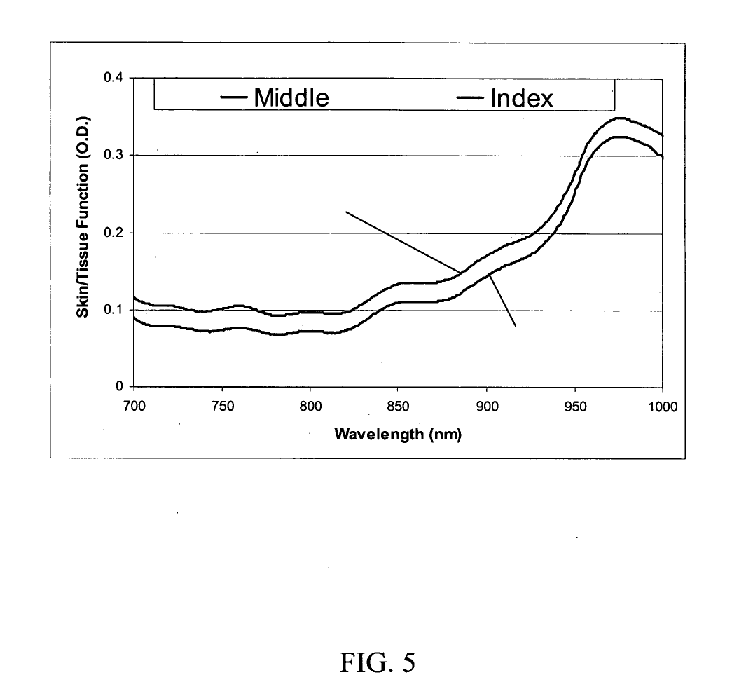

FIG. 5 shows two skin/tissue functions for a human subject. Measurements are made on index finger 510 and middle finger 520, respectively. It is clear that two skin/tissue functions have different strengths due to measured at two different sites. For a given skin/tissue function, the response varies with wavelength. These characteristics provide quantitative information to correct Raman signals, from which an accurate measurement of analyte concentration can be made.

Although the present invention has been described in terms of specific embodiments it is anticipated that alterations and modifications thereof will no doubt become apparent to those skilled in the art. It is therefore intended that the following claims be interpreted as covering all such alterations and modifications as fall within the true spirit and scope of the invention.

We claim:

1. An apparatus for non-invasively evaluating constituents under the skin of a patient comprising:
a first measurement module including a light source for illuminating the target tissue and a first detector for monitoring the optical response of the tissue, said measurement module being selected from the group consisting of Raman spectroscopy, infrared spectroscopy, infrared spectroscopy, fluorescence spectroscopy, polarization changes and scatter changes;
a second measurement module including a broadband light source for illuminating the target tissue and a second detector for monitoring the optical response of the tissue; and
a processor for receiving the output from said first and second detectors and operating to calculate a reflectance spectrum based on the output from the second detector and using that spectrum to correct the output from the first detector when evaluating the constituents in the patient.

2. An apparatus as recited in claim 1, wherein said first and second detectors are the same.

3. An apparatus as recited in claim 1, further including an optical path for monitoring the intensity of the light emitted from the broadband light source and wherein said processor uses that information to normalize measurements of the second detector.

4. An apparatus as recited in claim 1, wherein said first measurement module is configured to perform Raman spectroscopy.

5. An apparatus as recited in claim 4, wherein the illumination power of the broadband light source is below the Raman threshold.

6. An apparatus as recited in claim 5, wherein the processor functions to evaluate the glucose level in blood.

7. An apparatus for non-invasively evaluating the constituents in the blood of a patient comprising:
a laser light source for selectively illuminating the target tissue with narrow band optical radiation;
a broadband light source for selectively illuminating the target tissue with broadband optical radiation;
a spectrometer detector for monitoring the Raman spectral response from the target tissue when illuminated by the laser light source and for monitoring the spectral reflectance of the target tissue when illuminated by the broadband light source; and
a processor for using the monitored spectral reflectance to correct the Raman spectral response and for evaluating the constituents in the blood using the corrected Raman spectral response.

8. An apparatus as recited in claim 7, further including an optical path for monitoring the intensity of the light emitted from the broadband light source and wherein said processor uses that information to normalize measurements of the broadband light reflected from the target tissue.

9. An apparatus as recited in claim 8, wherein the illumination power of the broadband light source is below the Raman threshold.

10. An apparatus as recited in claim 9, wherein the processor functions to evaluate the glucose level in blood.

11. An apparatus as recited in claim 7, wherein the wavelength range of the broadband light source includes the wavelength emitted by the laser light source and at least a portion of the Raman shifted wavelengths.

12. An apparatus for non-invasively evaluating the constituents in the blood of a patient comprising:
a laser light source;
a broadband light source;
a spectrometer detector;
optics for selectively directing the laser light to the target tissue and the Raman spectral response to the detector, said optics for selectively directing the broadband light to the target tissue and the reflected light to the detector, said optic also selectively directing the broadband light to the detector without reflecting off the target tissue; and
a processor for generating a normalized reflectance spectrum using both measurements of the broadband light and using the normalized reflectance spectrum to correct the Raman spectral response and for evaluating the constituents in the blood using the corrected Raman spectral response.

13. An apparatus as recited in claim 12, wherein the illumination power of the broadband light source is below the Raman threshold.

14. An apparatus as recited in claim 13, wherein the processor functions to evaluate the glucose level in blood.

15. An apparatus as recited in claim 12, wherein the wavelength range of the broadband light source includes the wavelength emitted by the laser light source and at least a portion of the Raman shifted wavelengths.

16. A method for non-invasively evaluating the constituents in the blood of a patient comprising the steps of:
illuminating the target tissue with broadband light;
monitoring the spectral response of the tissue to the broadband illumination;
illuminating the target tissue with narrowband light;
monitoring the Raman spectral response to the narrowband light;
correcting the Raman spectral response using the monitored spectral response to the broadband light; and
evaluating the constituents in the blood using the corrected Raman spectral response.

17. A method as recited in claim 16, further including the steps of:
measuring the spectral intensity of the broadband light incident on the target tissue; and
normalizing the spectral response of the tissue to broadband light with the measurement of the spectral intensity of the incident broadband light.

18. A method as recited in claim 16, wherein the illumination power of the broadband light source is below the Raman threshold.

19. A method as recited in claim 16, wherein the constituent is glucose.

20. A method as recited in claim 16, wherein the wavelength range of the broadband light source includes the wavelength emitted by the laser light source and at least a portion of the Raman shifted wavelengths.

21. An apparatus for non-invasively evaluating the constituents in the blood of a patient comprising:
a laser light source for selectively illuminating the target tissue with narrow band optical radiation;
a second light source for selectively illuminating the target tissue with optical radiation having at least one wavelength;
a spectrometer detector for monitoring the Raman spectral response from the target tissue when illuminated by the laser light source and for monitoring the spectral reflectance of the target tissue when illuminated by the light from the second source; and
a processor for using the monitored spectral reflectance to correct the Raman spectral response and for evaluating the constituents in the blood using the corrected Raman spectral response.

22. An apparatus as recited in claim 21, further including an optical path for monitoring the intensity of the light emitted from the second light source and wherein said processor uses that information to normalize measurements of the light from the second source reflected from the target tissue.

23. An apparatus as recited in claim 21, wherein the illumination power of the light from the second source is below the Raman threshold.

24. An apparatus as recited in claim 21, wherein the processor functions to evaluate the glucose level in blood.

25. An apparatus as recited in claim 21, wherein the wavelength emitted by the second source is selected to either match the narrow band output of the laser light or fall within the range of the Raman shifted wavelengths.

26. An apparatus as recited in claim 21, wherein said second light source is arranged to emit a plurality of distinct wavelengths and wherein the Raman spectral response is corrected using measurements at more than one wavelength.

27. A method for non-invasively evaluating the constituents in the blood of a patient comprising the steps of:
illuminating the target tissue with at least one wavelength of light at a power less than the Raman threshold;
monitoring the response of the tissue to the reflected light;
illuminating the target tissue with narrowband light having a power higher than the Raman threshold;
monitoring the Raman spectral response to the narrowband light;
correcting the Raman spectral response using the monitored response to the light having a power less than the Raman threshold; and
evaluating the constituents in the blood using the corrected Raman spectral response.

28. A method as recited in claim 27, further including the steps of:
measuring the incident intensity of the light which was at a power less than the Raman threshold; and
normalizing the response of the tissue of the reflected light based on the measurement of the intensity of the incident light.

29. A method as recited in claim 27, wherein the constituent is glucose.

30. A method as recited in claim 27, wherein the wavelength of the light having a power less than the Raman threshold is selected to either match the narrow band light having a power higher than the Raman threshold or fall within the range of the Raman shifted wavelengths.

31. An apparatus as recited in claim 27, wherein the target tissue is illuminate with a plurality of distinct wavelengths each having a power less than the Raman threshold and wherein the Raman spectral response is corrected using measurements at more than one wavelength.
